(19) United States
(12) Patent Application Publication
(10) Pub. No.: US 2022/0148707 A1
(43) Pub. Date: May 12, 2022

(54) SYSTEMS AND METHODS FOR TIME-SENSITIVE ADAPTIVE TREATMENT OF MENTAL HEALTH CONDITIONS

(71) Applicant: Oui Therapeutics, LLC, New Haven, CT (US)

(72) Inventor: Seth Feuerstein, Woodbridge, CT (US)

(73) Assignee: Oui Therapeutics, LLC, New Haven, CT (US)

(21) Appl. No.: 17/521,784

(22) Filed: Nov. 8, 2021

Related U.S. Application Data
(60) Provisional application No. 63/111,562, filed on Nov. 9, 2020.

(51) Int. Cl. G16H 20/70; G16H 10/60; G16H 80/00; G16H 50/20; G16H 50/30

(52) U.S. Cl. CPC G16H 20/70; G16H 10/60; G16H 50/30; G16H 50/20; G16H 80/00

(57) ABSTRACT

Described herein are computer-implemented techniques for delivering and administering adaptive, personalized care to patients suffering from mental disorders and illnesses (including those creating a risk of suicide). Some aspects described herein provide a computer-implemented method for adapting treatment for a patient based on patient data, and administering the adapted treatment to the patient. For example, a patient's device may obtain the patient data and adapt and administer the treatment. Some aspects described herein provide a system for delivering adaptive treatment of mental disorders and illnesses over a communication network to one or more devices. Some aspects described herein provide a computer-implemented method for administering treatment activities to treat a patient who is at risk of dying by suicide. For example, a patient's device (e.g., mobile phone, tablet, computer, etc.) may select and administer one or more treatment activities to reduce the patient's risk of suicide.

### BACKGROUND

Several therapy methods are used to treat mental health conditions, such as disorders and illnesses, and to prevent related patient harm such as suicide. Our society is increasingly becoming aware of the need for better and more available therapy methods as people become more comfortable talking about their struggles and need for therapy, and as statistics show an alarming rise in suicide rates and other ill effects.

### RELATED APPLICATIONS

This application claims the benefit under 35 U.S.C. § 119(e) of U.S. Provisional Patent Application Ser. No. 63/111,562, filed on Nov. 9, 2020 and entitled “SYSTEMS AND METHODS FOR TIME-SENSITIVE ADAPTIVE TREATMENT OF MENTAL HEALTH CONDITIONS,” which is hereby incorporated herein by reference in its entirety.

### SUMMARY

Some aspects of the present disclosure provide a non-transitory computer-readable storage medium having encoded thereon instructions that, when executed by at least one processor, cause the at least one processor to carry out a method, the method comprising obtaining patient data related to a mental condition of a patient, wherein the patient data indicates timing information related to previously administered treatment for the mental condition of the patient; adapting, based on the patient data, a treatment for the mental condition of the patient, including adapting a rate or order of the treatment; and administering, to the patient, the treatment.

In some embodiments, the timing information related to the previously administered treatment indicates that, within 48 hours before administering the treatment to the patient, the patient received a previous treatment from a previous treatment list, the previous treatment list including: electroconvulsive therapy; intravenous ketamine; esketamine; a ketamine derivative; N-Methyl-D-aspartate; glutamate modulating entity; psychedelic drugs; and repetitive transcranial magnetic stimulation.

In some embodiments, the timing information related to the previously administered treatment indicates the previous progression of the patient through the previously administered treatment; and adapting the rate or order of the treatment comprises adapting the rate or order of the treatment based on the previous progression of the patient through the previously administered treatment.

In some embodiments, the treatment addresses suicidal tendencies of the patient.

In some embodiments, obtaining the patient data comprises asking the patient whether the patient is ready for a treatment activity, and adapting the treatment comprises selecting the treatment activity from a list of treatment activities.

In some embodiments, obtaining the patient data comprises obtaining sensory data from one or more sensors of a device of the patient, and the patient data indicates a response of the patient to previously administered treatment.

In some embodiments, obtaining the patient data comprises obtaining, over a communication network, instructions for selecting a treatment activity from the list of treatment activities, and adapting the treatment comprises selecting the treatment activity.

In some embodiments, the method further comprises transmitting, over the communication network to the healthcare provider, an indication of the patient's response to the treatment activity.

In some embodiments, adapting the rate or order of the treatment comprises: selecting, from an ordered list of treatment activities, at least one first treatment activity, rather than selecting at least one second treatment activity listed before the at least one first treatment activity in the ordered list; and selecting, at a later time, the at least one second treatment activity.

In some embodiments, obtaining the patient data comprises accessing an application on a device of the patient and determining a risk of suicide of the patient based on one or more of words spoken by the patient and/or a message sent by the patient.

In some embodiments, accessing the application comprises determining a contact of the patient, and the method further comprises sending, to the contact, a message.

Some aspects of the present disclosure provide system comprising at least one processor configured to, based on information indicating the patient received, within 48 hours before a treatment activity of a list of treatment activities to be administered to a patient is administered to the patient, a separate treatment from an additional list: adapt, for a mental condition of the patient, the list of treatment activities to be administered to the patient; and send, over a communication network, to a device of the patient, treatment activity data indicative of the list of treatment activities.

In some embodiments, the additional list includes: electroconvulsive therapy; intravenous ketamine; esketamine; a ketamine derivative; N-Methyl-D-aspartate; glutamate modulating entity; psychedelic drugs; and repetitive transcranial magnetic stimulation.

In some embodiments, the at least one processor is configured to adapt the list of treatment activities to fit a duration of inpatient treatment of the patient.

In some embodiments, the at least one processor is further configured to obtain, over the communication network, from the device, patient data indicative of the patient's response to at least one treatment activity of the list of treatment activities, including previous progression of the patient through the at least one treatment activity; adapt, based on the patient data, the list of treatment activities, including adapting a rate or order of the list of treatment activities based on the previous progression of the patient; and send, over the communication network, to the device, an update to the treatment activity data.

In some embodiments, the at least one processor is further configured to access electronic health records of the patient and adapt the list of treatment activities based on the electronic health records.

In some embodiments, the at least one processor is further configured to send, to a healthcare provider of the patient, a message relating to the patient.

In some embodiments, the at least one processor is further configured to send, to a contact of the patient, a message relating to the patient.

Some aspects of the present disclosure provide a non-transitory computer-readable storage medium having encoded thereon instructions that, when executed by at least one processor, cause the at least one processor to carry out a method, the method comprising based on previous progression of a patient through a previous treatment, selecting at least one treatment activity from a first list, the first list including; and treating a suicidal patient by administering, to the patient, the at least one treatment activity within 48 hours of administering at least one other treatment from a second list. The list includes an interactive experience tracking module configured to track at least one metric related to behavior of the patient, instructions on modifying behavior of the patient, information regarding stimulus control, relaxation training, interactive multimedia content for paced breathing, progressive muscle relaxation, imagery-induced relaxation, and/or self-hypnosis, instructions on use of medication, and instructions on user monitoring of and adjustment of thoughts of the patient.

In some embodiments, the second list includes: electroconvulsive therapy; intravenous ketamine; esketamine; a ketamine derivative; N-Methyl-D-aspartate; glutamate modulating entity; psychedelic drugs; and repetitive transcranial magnetic stimulation.

In some embodiments, the at least one treatment activity is occurring within 36 hours of administering the at least one separate treatment.

In some embodiments, selecting the at least one treatment activity comprises selecting a cognitive behavioral therapy (CBT) step from the list, and treating the suicidal patient comprises administering the CBT step to the patient.

In some embodiments, the method further comprises receiving, over a communication network, the list.

In some embodiments, the method further includes generating a message template. In some embodiments, the method further includes adapting the message template to generate a message. In some embodiments, the method further includes sending, to the patient, the message. In some embodiments, sending the message includes sending the message on behalf of a healthcare provider of the patient. In some embodiments, sending the message on behalf of the healthcare provider includes sending the message in a name of the healthcare provider. In some embodiments, the message includes a request for the patient to provide a status update. In some embodiments, the message is signed by the healthcare provider.

In some embodiments, the method further includes recording a suicidal episode of the patient. In some embodiments, recording the suicidal episode may include capturing audio and/or video of the suicidal episode. In some embodiments, recording the suicidal episode may include a written narrative of the suicidal episode.

In some embodiments, the method further comprises sending, to a healthcare provider of the patient, a message.

In some embodiments, the message notifies the healthcare provider that the patient is at risk of suicide.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a block diagram of an exemplary system for delivering and providing personalized, adaptive care to one or more patients, according to some embodiments.

FIG. 1B is a block diagram of an exemplary configuration of the memory of the computer of FIG. 1A, according to some embodiments.

FIG. 1C is a front view of an exemplary device that may be included in the system of FIG. 1A, according to some embodiments.

FIG. 1D is a block diagram of an exemplary configuration of the memory of the device of FIG. 1C, according to some embodiments.

FIG. 2 is a front view of the exemplary device of FIG. 1C displaying a notification, according to some embodiments.

FIG. 3A is a flow chart illustrating an exemplary computer-implemented method for adapting and providing treatment to a patient suffering from a mental health condition, according to some embodiments.

FIG. 3B is a flow chart illustrating an exemplary method for generating and sending messages to a patient from a provider, according to some embodiments.

FIG. 4A is a flow chart illustrating an exemplary method for adapting and delivering personalized care to a patient's device, according to some embodiments.

FIG. 4B is a flow chart illustrating an exemplary computer-implemented method for treating a patient who is at risk of dying by suicide, according to some embodiments.

FIG. 5 is a block diagram of the system of FIG. 1A further illustrating interactivity between a patient, a contact of the patient, and the patient's provider via the system.

FIG. 6 illustrates an example of a computing system environment with which some embodiments may operate.

### DETAILED DESCRIPTION

The inventor has developed computer-implemented techniques for treating one or more mental health conditions in a patient by administering personalized, adaptive care specific to the patient. In some embodiments, techniques described herein provide a computer-implemented platform configured to adapt treatment for a patient's mental health condition(s) based on patient data, and administer the treatment to the patient using an electronic device (e.g., a mobile device owned by or loaned to the patient). In some embodiments, systems and devices described herein may be configured to adapt treatment for a patient by adjusting the order, content, and pace at which treatment activities are delivered to a patient. According to various examples, patient data may include diagnosis data from the patient's healthcare provider and/or clinician, electronic health records of the patient, and/or data collected from the patient via the patient's electronic device (e.g., in real time). In some embodiments, mental health conditions addressed by techniques described herein may include suicide, insomnia, panic disorder, major depressive disorder, panic, phobias, obsessive compulsive disorder (OCD), treatment-resistant depression, irritable bowel syndrome, generalized anxiety, autism, pain syndromes, alone or in combination. Techniques described herein improve patient access to high quality treatment for such conditions at least in part by expanding treatment delivery beyond inpatient clinics and making the treatment process faster and/or more effective.

The inventor has recognized that one mental health treatment modality is Cognitive Behavioral Therapy (CBT), which is a type of psycho-social intervention to address problematic cognitive distortions (e.g., thoughts or attitudes) by developing coping strategies specific to the distortions. In contrast to psychoanalytic approaches, which look for an unconscious meaning behind the cognitive distortions, CBT aims to treat specific cognitive distortions that are symptomatic of a diagnosed mental health condition. As an example, a person who suffers from some mental health condition(s) may exhibit suicidal thoughts, and an example CBT treatment might be to distract the person from their suicidal thoughts.

The inventor has recognized that outpatient treatment for mental health conditions is conventionally administered on a weekly or monthly basis, which can limit the pace and content of the patient's prescribed treatment. For example, a patient may receive treatment at a weekly session and spend the following week practicing a single treatment exercise before meeting with the patient's therapist again. As a result, patients who are able to complete treatment exercises faster are not able to make additional treatment progress before meeting with the patient's therapist the following week. Moreover, for patients with multiple mental health conditions, a therapist may only prescribe one treatment exercise for a single condition per week, whereas a patient may have time to complete multiple treatment exercises for multiple conditions during that time.

The inventor has also recognized that the weekly or monthly outpatient treatment sessions are too spaced out to be administered in an inpatient setting. For example, when a patient is diagnosed with one or more mental health conditions, the patient's healthcare provider or clinician may prescribe outpatient treatment such as CBT at weekly or monthly meetings with a therapist. As a result, the typical outpatient treatment timeline is too long to be implemented in an inpatient setting, where patients may often reside in a clinic or hospital for a week or two at most. As a result, inpatient treatment usually relies on one-size-fits-all treatments such as group therapy, which do not provide patients with the individually focused treatments provided by long term CBT. For example, weekly face-to-face meetings with a therapist can allow the therapist to get to know the patient's unique situation and to adapt treatment to the patient's specific symptoms, personality, lifestyle, etc. In addition, patients receiving inpatient group therapy may have to spend time working on addressing problems they do not personally have, thus wasting valuable time during a short inpatient stay. For example, a patient who does not have a sleeping problem may spend time in a group therapy session learning about strategies for sleeping, rather than learning how to react to their suicidal thoughts. Moreover, such inpatient treatment methods do not include any follow up measures to check in on patients post-discharge.

The inventor has also recognized that inpatient settings typically do not have therapists on-site who specialize in every mental condition from which patients may be suffering. Accordingly, treatment for one or more of a patient's mental conditions may be unavailable during an inpatient stay.

The inventor has also recognized that conventional computer-implemented methods for therapy are not comprehensive, provide few features, and are passive. For example, conventional methods may provide a generic questionnaire and do not actively interact with the patient.

To address these problems, the inventor has developed techniques for delivering personalized, adaptive care to patients suffering from mental health conditions. In some embodiments, a computer-implemented method for treating patients suffering from one or more mental health conditions may include obtaining patient data related to the patient's mental condition(s), adapting treatment for the patient's medical condition(s) based on the patient data, and administering the adapted treatment to the patient. For example, the method may be performed by a device of the patient. In some embodiments, the treatment may address multiple mental health conditions of the patient simultaneously, sequentially, and/or in an interspersed order. In one example, the treatment may address the patient's risk of dying by suicide. Some embodiments provide a system for delivering adaptive treatment of mental health conditions over a communication network to one or more devices. For example, the system may adapt a list of treatment activities for a patient suffering from a particular mental health condition and send the adapted list to the patient's device.

In some embodiments, obtaining the patient data may include asking the patient whether the patient is ready for a particular treatment activity. In some embodiments, obtaining the patient data may include obtaining sensory data from sensors of the patient's device. The sensory data may indicate the patient's readiness for a particular treatment activity, and/or the patient's response to previously administered treatment, such as the patient's level of fatigue and/or attentiveness to the previously administered treatment. Alternatively or additionally, obtaining the patient data may include monitoring activity on the patient's device (e.g., content of text messages, emails, phone calls, and social media posts, or playing certain songs and/or videos, etc.), which may indicate whether the patient is ready for a particular treatment activity.

In some embodiments, obtaining the patient data may include obtaining instructions (e.g., over a network) for selecting a treatment activity. For example, the instructions may be specific to the patient, such as issued by the patient's healthcare provider. In some embodiments, the method may include sending (e.g., over the network) an indication of the patient's response to the treatment activity (e.g., from patient input and/or sensory data) to the healthcare provider. Alternatively or additionally, instructions may be automatically generated by a system having access (e.g., over the network) to the patient's electronic health records. Accordingly, treatment may be personalized based on data obtained from the patient and/or from the patient's provider or electronic health records. In some embodiments, the patient data may indicate the patient's treatment progress from an inpatient stay, and the method includes selecting a treatment activity determined based on the patient's progress from the inpatient stay. Accordingly, in some embodiments, techniques described herein may provide a more seamless transition from inpatient to outpatient treatment.

Adapting the treatment based on the patient data may include selecting a treatment activity to administer from a list of treatment activities. For example, if the patient data indicates that the patient is ready for a particular treatment activity, the treatment may be adapted for the patient by selecting the treatment activity from the list. Alternatively, if the patient is not ready, the treatment may be adapted by not selecting the treatment activity. In some embodiments, the treatment activities may be organized in an ordered list (e.g., by order of administration) and the treatment may be adapted by changing the order of the list based on the patient data. For example, activities on the list may be swapped in order, and/or some activities may be repeated or omitted. In cases where instructions for selecting a treatment activity are received, adapting the treatment may include selecting the treatment activity based on the instructions. The inventor has recognized that by adapting treatment to a particular patient based on patient data, the timeline for administering treatment may be reduced to fit a particular duration, such as the duration of an inpatient stay. It should be appreciated that, alternatively or in addition to inpatient treatment, such methods may deliver outpatient treatment. Examples of treatment activities that may be administered according to techniques described herein include: psychoeducational material, clinical vignettes, questionnaires, cognitive exercises, behavioral exercises, challenging thoughts, A-B-C exercises, safety planning, crisis response planning, exposure, imagined exposure, sleep diary creation and/or management, interactive fill-in content, and others.

In some embodiments, the method includes determining and/or learning to determine the patient's status. For example, by monitoring the patient's activity (e.g., using sensors and/or by detecting activity on applications on the device) a determination can be made as to the patient's response to treatment activities. In one example, a patient's attentiveness to treatment activities may be determined by eye-tracking and/or the rate at which the patient completes a treatment activity. In one example, the method may include prompting the patient to decide whether to pause treatment and resume at a later time (e.g., a few hours later). In some embodiments, data indicating the patient's status may be used to further adapt treatment, such as by accelerating or slowing down the delivery of treatment activities in response to the time taken by the patient to complete previous treatment activities. In some embodiments, data indicating the patient's status may be input to a trained model configured to determine a treatment pace that will be effective for the patient based on the status data. In one example, a patient may complete one treatment activity faster than another treatment activity, and the patient may not complete a third treatment activity. In this example, a trained model may use data indicating the patient's rate of completion (or incompletion) and data pertaining to the exercises previously administered to reorder a list of treatment activities such that activities the patient is likely to complete are provided first and activities the patient is unlikely to complete are saved for later or removed from the list. Alternatively or additionally, in this example, activities may be reordered based on suitability of the exercise to patient response and/or inpatient or outpatient setting, such as delivering more intense activities sooner and delaying safety activities in an inpatient setting.

The inventor has also developed systems for delivering personalized, adaptive care to patients suffering from mental illness, such as to the patient's device(s) over a network (e.g., the Internet). In some embodiments, a system may include a processor (e.g., within a computer) configured to adapt a list of treatment activities for a patient having a particular mental condition, and to send treatment activity data indicative of the list of treatment activities to the patient's device over the network. In some embodiments, the processor may be configured to adapt the list of treatment activities to fit a particular duration of treatment. For example, the list may be adapted to fit the duration of a patient's inpatient stay. The inventor has recognized that by adapting treatments to the patient's mental condition and/or the duration of the inpatient stay, patients may receive personalized treatment that is typically unavailable in inpatient settings due to the short duration of the stay and the lack of specialist or dedicated therapists. It should be appreciated that, alternatively or in addition to inpatient treatment, such systems may deliver outpatient treatment.

In some embodiments, the processor may also be configured to obtain (e.g., over the network) patient data indicative of the patient's response to the treatment activities. For example, the patient data may be obtained from the patient's device. The processor may adapt the list of treatment activities based on the treatment data and send an update to the treatment activity data (e.g., over the network) to the patient's device. For example, upon determining that a patient is progressing through treatment activities at a faster rate than expected, the treatment activity data may be updated to reflect the increased number of treatment activities the patient may receive in the duration of the patient's inpatient stay. In some embodiments, the processor may be configured to send a message (e.g., over the network) to the patient's healthcare provider relating to the patient. For example, the message may indicate the patient's progress or lack thereof such that the healthcare provider may respond with instructions for further adapting the list of treatment activities. In some embodiments, the processor may be configured to send a digital or hard copy letter to the patient on behalf of the patient's healthcare provider, such as to check on the patient, and/or to follow up with the patient after the patient completes treatment. In some embodiments, the processor may be configured to obtain (e.g., from the patient's device) contact information for a contact of the patient (e.g., a friend or family member) and/or to reach out to the contact on behalf of the patient. For example, the processor may be configured to send a message to and/or call the contact to request that the contact get in touch with the patient.

In some embodiments, progression of the patient may include some measure of how well the patient is doing with the therapy. In some embodiments, the measure may be cognitive and/or behavioral. For example, progression may include how much the patient is practicing the therapy (such as CBT), how much they are integrating it into their behavior and/or speech, and so on.

In some embodiments, the processor may be configured to access the patient's electronic health records, such as over the network, and to adapt the list of treatments based on the electronic health records. For example, the electronic health records may indicate the patient's medical condition such that the list of treatments may be adapted to that particular medical condition. Alternatively or additionally, the electronic health records may indicate the patient's response to previous treatments or lack of previous treatments such that appropriate care and/or precautions may be taken when generating the list of treatments. In some embodiments, the processor may be configured to receive (e.g., over the network) information from the patient's healthcare provider such that the list of treatments may be adapted based on the healthcare provider's input.

Some aspects described herein provide computer-implemented techniques for treating patients suffering from suicide, such as a computer-implemented method for administering treatment activities to treat a patient who is at risk of dying by suicide. For example, a patient's device (e.g., mobile phone, tablet, computer, etc.) may be configured to select and administer one or more treatment activities to reduce the patient's risk of suicide.

The inventor has recognized that mental health conditions are primarily treated by physicians, psychologists, or masters-level mental health social workers, who are not usually available at night, which is when some patients (e.g., suicidal patients) may need the most help. This presents a problem for clinicians responsible for the care of suicidal patients. In addition, conventional approaches for suicide prevention rely on patients to contract for their own safety, which has been shown to be ineffective in preventing further suicide attempts, and/or fill out a questionnaire for safety planning. These methods have drawbacks in that they rely on the patient to be honest and self-aware enough to provide accurate information, and also in that the questionnaire is usually the same for all patients, thus failing to take into account any information already known and specific to the patient.

The inventor has also recognized that recent improvements in the treatment of mental health conditions (like depression) have not translated to a reduction in suicide attempts in patients who receive the improved treatments. For instance, patients diagnosed with mental health conditions like depression may receive acute treatment in the form of electroconvulsive therapy (ECT), intravenous ketamine, esketamine (e.g., SPRAVATO®), any ketamine derivative or N-Methyl-D-aspartate (NDMA), glutamate modulating entity, psychedelic drugs (e.g., LSD), repetitive transcranial magnetic stimulation (rTMS), or other treatments with high quality or widespread access. The inventor has recognized that while at least some of these acute treatments tend to show a reduction in symptoms of mental health conditions like depression in patients immediately after treatment, such as for a few days, patients often (seemingly counter-intuitively) attempt suicide in the weeks and months after treatment. In many cases, patients are actually more likely to attempt suicide in these post-treatment weeks and months than if the treatments were not administered at all.

The inventor has recognized that there has conventionally been a misunderstanding of suicide attempts and suicide risk. The inventor has recognized that suicide risk and symptoms of mental health conditions like depression are not directly linked and that suicide risk (especially during such post-treatment time) may be reduced independently of such symptoms, such as by using some embodiments herein during, immediately after, or within some range of time after treatment such as the acute treatments described herein.

In response to these and other issues, the inventor has developed therapeutic modalities which incorporate computer-implemented techniques for administering treatment activities to treat a patient, including patients who are at risk of dying by suicide. In some embodiments, a computer-implemented method for treating a patient who is at risk of suicide includes selecting a treatment activity from a list of treatment activities and administering the treatment activity to the patient. The list may include at least one of: an interactive experience tracking module configured to track at least one metric related to behavior of the patient; instructions on modifying behavior of the patient; information regarding stimulus control; relaxation training; interactive multimedia content for paced breathing, progressive muscle relaxation, imagery-induced relaxation, and/or self-hypnosis; instructions on use of medication; and/or instructions on user monitoring of and adjustment of thoughts of the patient). For example, the method may be performed by a patient's device. In some embodiments, the treatment activity may be a cognitive behavioral therapy (CBT) step to be administered. Alternatively, the method may deliver other treatment activities or therapies to the patient.

In some embodiments, the method includes obtaining patient data, manually or automatically. For example, the method may include asking the patient how the patient feels and/or whether the patient needs help. Alternatively or additionally, the method may include detecting a risk of suicide of the patient, such as through a sensor of the patient's device (e.g., camera, accelerometer, microphone, etc.) or by monitoring activity on the patient's device (e.g., content of text messages, emails, phone calls, and social media posts, or playing certain songs and/or videos, etc.). For example, thoughts and/or behavior may be monitored in such ways. In one example, the patient's risk of dying by suicide may be determined based on monitoring patient activity, such as by determining and storing certain activities that may be unique to the patient (e.g., signature activities) for later use in determining the patient's status. Other information may be determined as well, such as a contact of the patient (e.g., a friend or family member). In the event that the patient is at increased risk of suicide, or if it is determined that the patient would benefit from interacting with the contact, the method may include reaching out to the contact (e.g., sending a message or initiating a phone call) on behalf of the patient to request that the contact get in touch with the patient. In some embodiments, the method includes sending a digital or hard copy letter to the patient from the patient's provider, such as to check on the patient, and/or to follow up with the patient after the patient completes treatment.

In some embodiments, the method includes receiving treatment activity data over a communication network, such as the Internet. For example, the treatment activity data may be provided over the communication network to the device from the patient's healthcare provider such as the patient's doctor. In some embodiments, the method may include sending a message to the healthcare provider of the patient. For example, the method may notify the healthcare provider that the patient is at risk of suicide. Alternatively or additionally, the method may provide a status update to the healthcare provider regarding the patient, such as a report of recent activity by the patient.

In some embodiments, systems and methods described herein may be implemented (e.g., used to treat the patient using at least one treatment activity) during or shortly after administration of separate treatment(s) of mental health condition(s) like depression. For example, such separate treatment(s) may include acute treatments like ECT, intravenous ketamine, esketamine, any ketamine derivative or NDMA, glutamate modulating entity, psychedelic drugs, rTMS, or other treatments with high quality or widespread access. For example, systems and methods described herein may be implemented during such separate treatment(s) or within 24-48 hours (e.g., within 36 hours) of the separate treatment(s) or similar treatments. For example, in some embodiments any computer-implemented treatment for preventing suicide described herein (such as selecting treatment activity from a list including CBT; administering personalized, adaptive treatment; and/or coordinating treatments for patients, connecting patients to providers, connecting patients to contacts, and adapting treatment based on information from healthcare providers and the patients' electronic health records) may be used in combination with acute treatments like ECT, intravenous ketamine, esketamine, any ketamine derivative or NDMA, glutamate modulating entity, psychedelic drugs, rTMS, or other treatments with high quality or widespread access.

By providing computer-implemented treatment for preventing suicide, such as using a patient's device, patients may receive treatment and suicide prevention protocols may be initiated even when clinicians are unavailable.

It should be appreciated that aspects of systems and methods described herein may be implemented alone or in combination. In addition, such systems and methods may be used to treat mental disorders and illnesses other than those creating a risk of suicide.

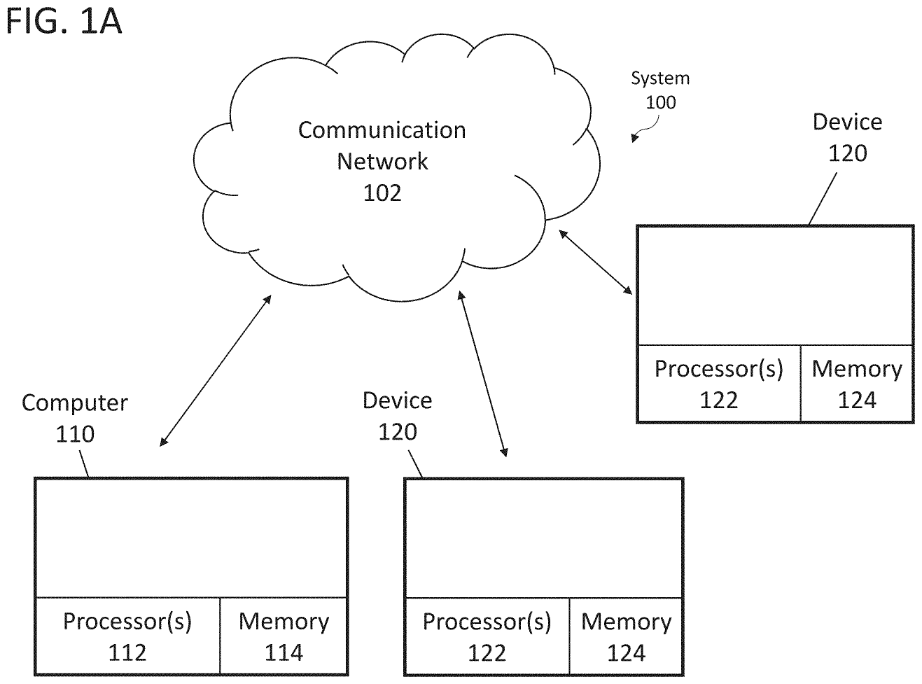

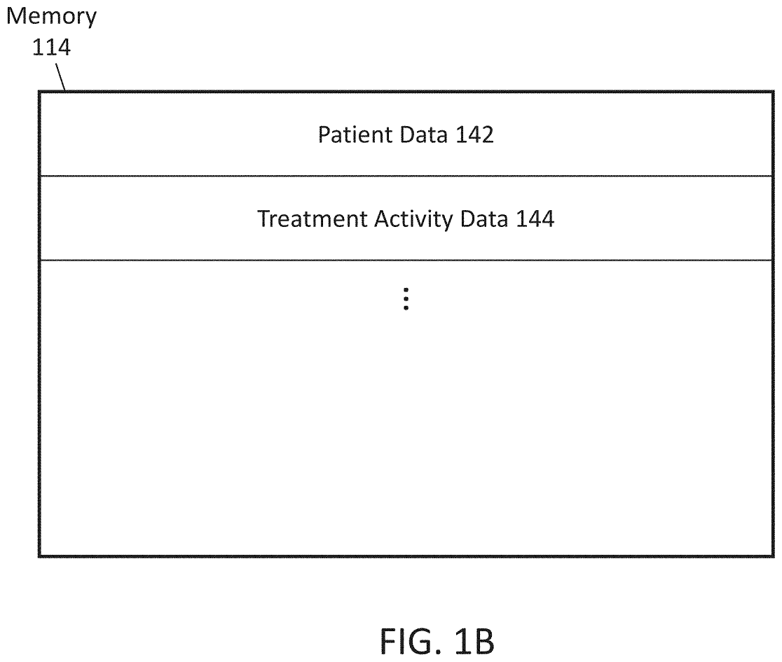

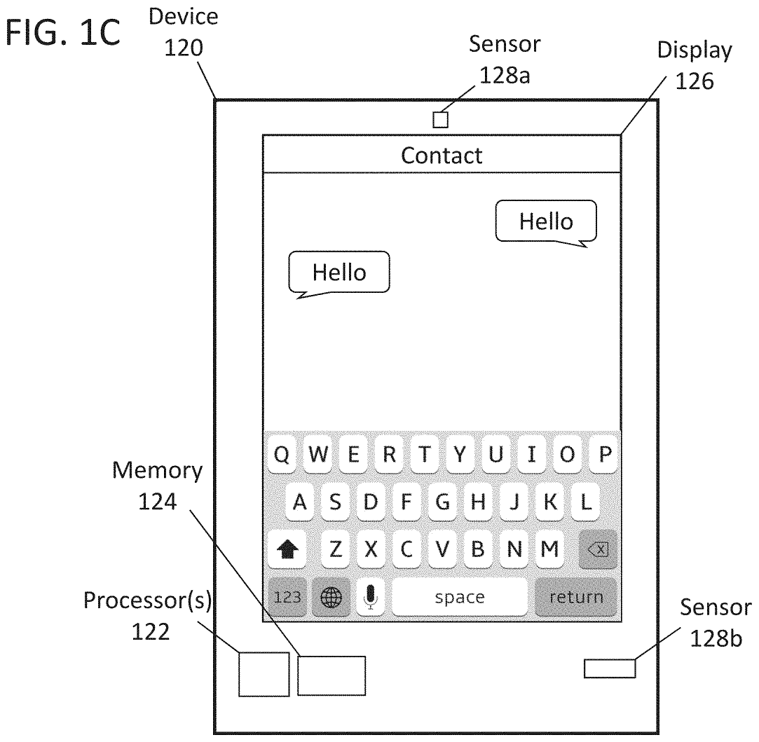

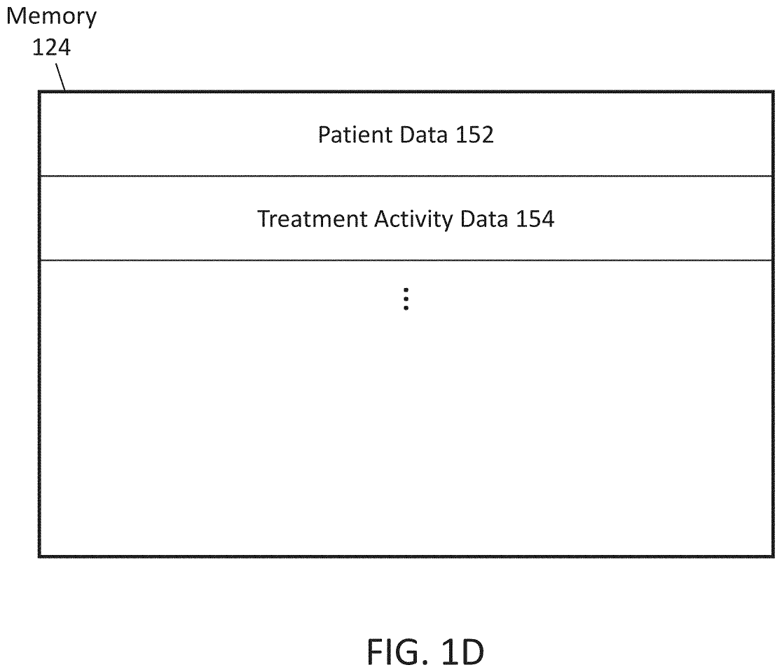

FIG. 1A is a block diagram of exemplary system 100 for delivering and providing personalized, adaptive care to one or more patients, according to some embodiments described herein. System 100 includes computer 110 and devices 120, which may be configured to communicate with one another over communication network 102. In some embodiments, computer 110 may be configured to provide a list of selected treatment activities to be administered to a patient and send the list to one or more of devices 120 to be administered. In some embodiments, the selected treatment activities may include processor-executable instructions that, when executed, cause device(s) 120 to deliver audio/visual treatment content to the patient, as described further herein. In some embodiments, computer 110 may be configured to select the treatment activities based on patient data (e.g., diagnosis data) stored in memory 114. In some embodiments, computer 110 may be further configured to adapt the selected treatment activities based on patient data (e.g., patient response data) received via device(s) 120, as described further herein. It should be appreciated that, in some embodiments, computer 110 may be configured to provide treatment activates to device(s) 120 and device(s) 120 may be configured to select the treatment activities for administering to the patient.

In some embodiments, computer 110 may serve as a central hub configured to generate and provide treatment activities and/or patient data to device(s) 120. Computer 110 includes at least one processor 112 and a memory 114. In some embodiments, computer 110 may include one or more servers. In some embodiments, processor(s) 112 of computer 110 may be configured to generate treatment activity data using patient data stored in memory 114. For example, the treatment activity data may include a comprehensive list of treatment activities for a plurality of mental health conditions, the patient data may indicate the patient has one or more mental health conditions, and processor 112 may be configured to select a subset of the treatment activities for generating a list based on the mental health condition(s) of the patient. An exemplary configuration of memory 114 is illustrated in FIG. 1B.

FIG. 1B is a block diagram of exemplary configuration of memory 114 of computer 110, according to some embodiments. As shown in FIG. 1B, memory 114 stores patient data 142 and treatment activity data 144. In some embodiments, patient data 142 may include diagnosis data from the patient's healthcare provider and/or clinician indicating the patient's mental health condition(s). In some embodiments, patient data 142 may indicate that the patient was treated with ECT, intravenous ketamine, esketamine, any ketamine derivative or NDMA, glutamate modulating entity, psychedelic drugs, and/or rTMS treatment recently, such as within the last 24, 36, or 48 hours, or is currently doing so. Alternatively or additionally, in some embodiments, patient data 142 may include status data received via device(s) 120 indicating the patient's response to previously administered treatment activities, as described further herein. In some embodiments, treatment activity data 144 may include application data (e.g., processor-executable instructions and/or personalized application content, etc.) for a number of treatment activities from which processor(s) 112 may be configured to select for the patient. According to various embodiments, treatment activity data 144 may include application data for: psychoeducational materials, clinical vignettes, questionnaires, cognitive exercises, behavioral exercises, challenging thoughts, A-B-C exercises, safety planning, crisis response planning, exposure, imagined exposure, sleep diary creation and/or management and others. It should be appreciated that treatment activity data 144 may include multiple levels for the different treatment activities, with higher levels available for delivering to the patient once the patient has completed a lower level treatment activity from a same activity or category of activity.

In some embodiments, processor(s) 112 may be configured to switch an order in which selected treatment activities are to be administered, such as by reordering an ordered list of treatment activities in treatment activity data 144. In some instances, processor(s) 112 may be configured to add and/or remove treatment activities from treatment activity data 144. In some embodiments, processor(s) 112 may be configured to obtain at least some of patient data 142 and/or treatment activity data 144 over communication network 102, such as from the patient's electronic health records, the patient's healthcare provider (e.g., physician or therapist), and/or device(s) 120. In one example, processor(s) 112 may obtain additional treatment activity over communication network 102 to add to and/or replace treatment activity data 144. In some instances, a computer system associated with the patient's provider may provide at least some of treatment activity data 144 to computer 110 over communication network 102.

In some embodiments, devices 120 may be configured to receive a list of selected treatment activities from computer 110 for administering to the patient. As shown in FIG. 1A, each device 120 includes at least one processor 122 and a memory 124. In some embodiments, devices 120 may be patients' personal devices. For example, devices 120 may include mobile phones belonging to various patients. Alternatively or additionally, devices 120 may include multiple devices for each patient, such as a mobile phone and tablet computer, laptop computer, desktop computer, or other such devices. In some embodiments, devices 120 may include one or more passive monitoring devices in an inpatient unit. For example, the monitoring devices (e.g., cameras) may capture patient data and provide the patient data to computer 110 and/or other devices 120. It should be appreciated that, in some embodiments, devices 120 may be configured to receive patient data and a list of treatment activities from which devices 120 may be configured to select based on the patient data. An exemplary device 120 is further illustrated in FIG. 1C.

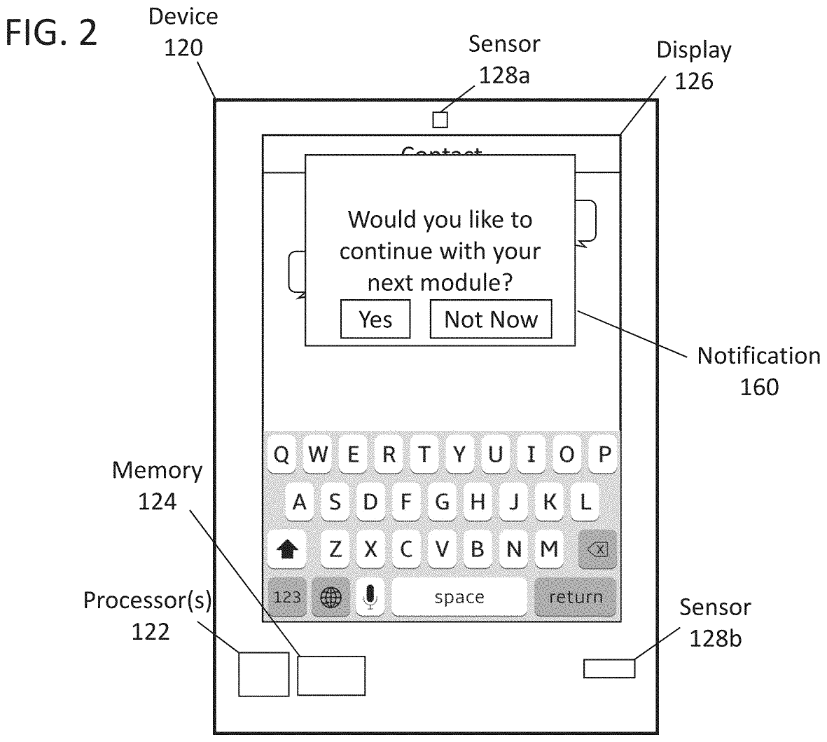

FIG. 1C is a front view of an exemplary device 120 of FIG. 1A, according to some embodiments. In FIG. 1C, device 120 is shown further including display 126 and sensors 128a and 128b. In some embodiments, device 120 may be configured to administer treatment activities to a patient and/or obtain patient data from the patient. In FIG. 1C, device 120 is illustrated as the patient's mobile phone. However, it should be appreciated that, in some embodiments, device 120 may include the patient's laptop and/or desktop computer, tablet computer, and/or other such devices. In some embodiments, display 126 may be configured to show application data, such as the messaging application illustrated in FIG. 1C, and/or display treatment activity notifications, such as illustrated in FIG. 2. An exemplary configuration of memory 124 of device 120 is illustrated in FIG. 1D.

FIG. 1D is a block diagram of an exemplary configuration of memory 124 of device 120, according to some embodiments. In FIG. 1D, memory 124 stores patient data 152 and treatment activity data 154. In some embodiments, patient data 152 and treatment activity data 154 may be received, at least in part, over communication network 102 from computer 110. In some embodiments, portions of patient data 152 may be obtained via sensors and/or application data from device 120. In some embodiments, processor(s) 122 of device 120 may be configured to administer treatment activities using treatment activity data 154. For example, treatment activity data 154 may include application data for a number of treatment activities selected by processor 112 of computer 110 to be administered to the patient. In some embodiments, treatment activity data 154 may include processor-executable instructions that cause processor(s) 122 to run treatment activity applications, or cause an application executing on processor(s) 122 to administer a particular treatment activity. In one example, executing an application may include displaying a questionnaire on display 126 with visual prompts for patient input by text and/or voice. In another example, executing an application may include displaying and/or playing audio of psychoeducational content, such as including instructions for the patient to perform a treatment exercise. In this example, executing the application may include collecting text, voice, and/or sensory feedback from the patient indicating the patient's response to the psychoeducational content. Exemplary execution of an application is described further including with reference to FIG. 2.

FIG. 2 is a front view of device 120 executing a treatment activity application, according to some embodiments. As shown in FIG. 2, display 126 of device 120 may display notifications such as notification 160 asking the patient whether the patient would like to conduct a treatment activity. Other notifications include prompts like “Now that you have completed module 1, would you like to practice the skills you have learned?” or “Now that you have completed module 1, would you like to schedule time later (at night) to continue with your next module?” In some embodiments, processor(s) 122 may be configured to display notifications based on patient data 152 and/or treatment activity data 154 stored in memory 124. In some embodiments, display 126 may include a liquid crystal display (LCD) or light emitting diode (LED) display screen. In some embodiments, display 126 may include a touchscreen. For example, as shown in FIG. 2, device 120 may be configured to respond to the patient touching the “Yes” or “Not Now” buttons displayed on display 126. In some embodiments, the patient's response to notifications may be saved in patient data 152 for use in adapting future treatment activities.

In some embodiments, display 126 may be configured to deliver treatment activity content visually and/or receive user input from the patient. For example, treatment activity content may be generated using treatment activity data 154 stored in memory 124. In some embodiments, display 126 may be configured to display video treatment activity content for the patient to watch. In some embodiments, display 126 may be configured to display a visual prompt for patient input, such as for audio, video, and/or text input. In one example, the prompt may ask for the patient's input as part of a treatment activity, or for the patient to provide information that may be used to adapt treatment.

In some embodiments, sensors 128a and/or 128b may be configured to capture patient input and/or feedback in connection with administered treatment activities. In some embodiments, sensor 128a may include a camera and/or microphone, and sensor 128b may include an accelerometer and/or a gyroscope. For example, the camera and/or microphone may be configured to record video and/or audio signals of the patient. The accelerometer and/or gyroscope may be configured to record movement of device 120, which may include recording movement of the patient. In some embodiments, device 120 may use recorded data from sensors 128a and/or 120b to determine the patient's risk status and/or availability for treatment activities.

In some embodiments, devices 120 may be configured to obtain patient data from the patient such that treatment activities can be adapted (e.g., by computer 110 and/or device 120) based on the patient data. For example, a device 120 may be configured to prompt the patient for input e.g., visually on a display and/or audibly using speakers or headphones), such as to ask whether the patient is ready for a treatment activity, and/or how the patient is feeling. Alternatively or additionally, processor(s) 122 may be configured to monitor one or more sensors of device 120 and/or one or more applications on device 120 for patient response data. For example, processor(s) 122 may be configured to determine the patient's response to currently and/or previously administered treatment activities and/or need for a particular treatment activity based on sound (e.g., speech) detected by a microphone of device 120 and/or motion detected by an accelerometer and/or gyroscope of device 120. Further examples of sensory data that may be used to determine patient response include eye tracking data, heart rate, blood pressure, pupillary dilation, facial expression, and others, which may be determined using a heart rate monitor, pulse oximeter, camera, and/or other such sensors. Alternatively or additionally, processor(s) 122 may make such a determination based on a text message, email, or social media post sent by the patient using device 120, and/or a song or video playing on device 120. In some embodiments, processor(s) 122 may be configured to execute natural language processing to determine the content of a text message, audio transcription, and/or the like.

In some embodiments, processor(s) 112 may be configured to send at least some of patient data 142 to device(s) 120, such that device(s) 120 may adapt a list of treatment activities stored on device 120 based on patient data 142. In some instances, device(s) 120 may be configured to receive updates from computer 110 to add to and/or replace treatment activity data stored on device(s) 120. For example, a list of treatment activities from the updated list may override treatment activities from the previous list.

In some embodiments, device(s) 120 and/or computer 110 may be configured to execute a model trained on data of any number of patients and configured to receive patient data as an input and output an indication of one or more treatment activities based on the patient data. In some embodiments, the trained model may employ supervised machine learning. For example, the trained model may be configured as a trained statistical classifier. In this example, the trained model may be trained using patient data and treatment activities identified by a clinician as being suitable for delivering to the patient based on the patient data. In one example, device 120 may be configured to monitor a suicidal patient's sleep (e.g., using sensor(s) 128a and/or 128b) and input patient data from monitoring to a trained model that is configured to output an indication of sleep improvement methodologies (e.g., sleep restriction and/or cognitive restructuring around sleep, etc.) as a selected treatment activity. In another example, device 120 may be configured to input patient response data into a trained model configured to output the patient's preferred time for delivering treatment. In this example, device 120 may be configured to prompt the patient at various times (e.g., visually or by audio) to ask if the patient would like to engage in a treatment activity, and patient responses to the prompts may be input to the trained model.

In some embodiments, device 120 may be configured to monitor phone and/or messaging applications executed on device 120 to obtain patient data 152. For example, device 120 may be configured to determine patient data 152 based on calls and text messages whether the patient is at an elevated risk level. Alternatively or additionally, device 120 may be configured to detect when the patient has not been contacted by one or more specified contacts, and automatically generate a notification in the device(s) of the specified contact(s).

In some embodiments, devices 120 may be configured to obtain patient data 152 from application data generated using previously administered treatment activities. For example, processor(s) 122 may be configured to record how long a patient took to complete a treatment activity, how focused the patient was during the treatment activity, and other such indications that may be determined from application and/or sensory data either alone or in combination. In this example, processor(s) 122 may display on device 120 a prompt for the patient asking whether the patient would like to pause treatment after application data indicates the patient took more than a threshold amount of time to complete a treatment activity, if processor(s) 122 determines the patient was substantially distracted during the treatment activity (e.g., based on eye tracking), and/or if processor(s) 122 determines the patient's heart rate was greater than a threshold level (e.g., based on a heart rate monitor). Alternatively, in this example, processor(s) 122 may display on device 120 a prompt for the patient asking whether the patient would like to proceed to another treatment activity after application data indicates the patient took less than a threshold amount of time to complete the treatment activity. In some embodiments, device 120 may be configured to transmit patient response data, including application and/or sensory data, and/or determinations made based on the application and/or sensory data, to computer 110 such that computer 110 may adapt the selected treatment activities for the patient based on the received application, sensory, and/or determination data.

In some embodiments, device 120 may be configured to deliver personalized treatment activity content to the patient, such as including audio and/or visual content based on patient input. The inventor has recognized that delivering personalized audio and/or visual content electronically to a patient via device 120 provides an unexpected therapeutic effect, as the audio and/or visual content triggers a unique response in the patient's brain. In one example, device 120 may be configured to administer a first treatment activity in which device 120 prompts the patient to input to device 120 a story that happened to the patient. In this example, the patient may input the story by video, audio, and/or text using device 120. Device 120 may be configured to administer a second treatment activity in which device 120 provides audio and/or visual content from the story to the patient. Without being bound by any particular theory, the brain can restructure and change its perception of what occurred by hearing and seeing content. The brain can also remember new details of what occurred, and even recognize patterns occurring in the future, thus preparing the brain to avoid bad activities. The inventor has recognized that while conventional approaches shielded patients from hearing or seeing their own stories out of fear it would destabilize or worsen their condition, techniques described herein may reset cognitive beliefs and modify future behavior, setting a resilience in the brain that reduces the likelihood of the patient's mental health condition worsening. In some embodiments, device 120 may be configured to indicate the risk level(s) of the patient throughout the story, such as in the form of a risk curve having points that refer to moments in the patient's story. In another example, device 120 may be configured to detect when the patient is at risk using sensory and/or application data, and to administer a treatment activity including the sensory and/or application data.

Communication network 102 may include a wired and/or wireless network over which computer 110 and devices 120 may communicate. In some embodiments, communication network 102 may also facilitate access to a patient's electronic health records, the patient's healthcare provider, and/or contacts of the patient. In some embodiments, communication network 102 may include the Internet. In some embodiments, communication network 102 may include a local area network (LAN), a wireless local area network (WLAN) such as Wi-Fi, a Bluetooth network, or other suitable networks.

It should be appreciated that, in some embodiments, memory 124 may be configured to store a list of treatment activities from which processor(s) 122 of each device 120 is configured to select treatment activities for administering to the patient.

It should be appreciated that, in some embodiments, computer 110 may include multiple memories 114. Alternatively or additionally, computer 110 may access memory 114 over communication network 110. In some embodiments, computer 110 may not serve as a central hub. For example, system 100 may be decentralized (e.g., distributed), and computer 110 may be one of devices 120. FIG. 1C is a front view of an exemplary device 120 that may be included in system 100, according to some embodiments. As shown in FIG. 1C, device 120 may be a tablet computer or phone having one or more processors 122, memory 124, display 126, and sensors 128a and 128b.

One example of delivering treatment activities to a patient at risk of suicide is described herein including with reference to FIG. 2. It should be appreciated that, according to various embodiments, treatment activities may or may not be adapted based on patient data before delivering to the patient.

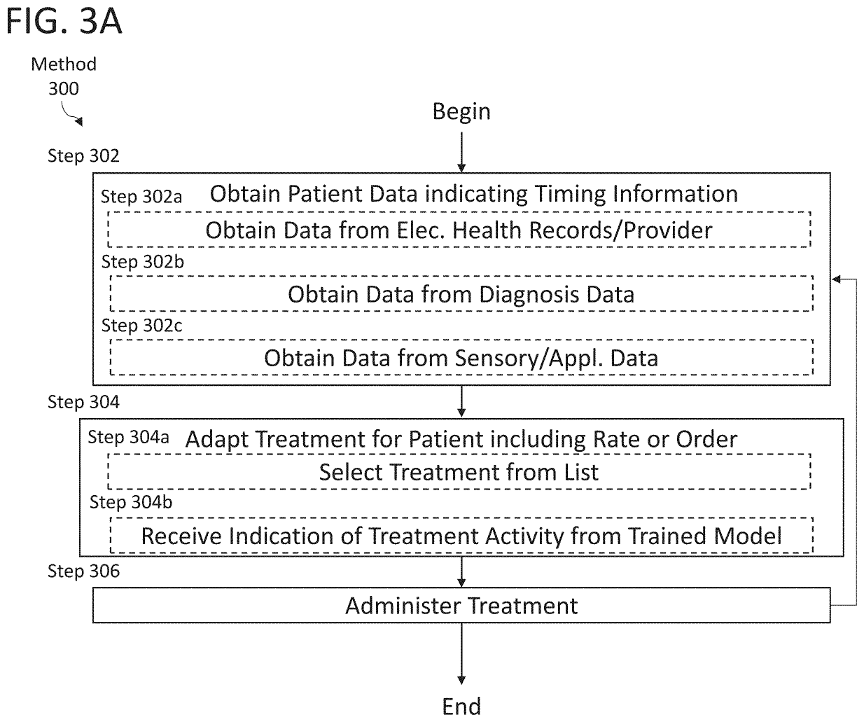

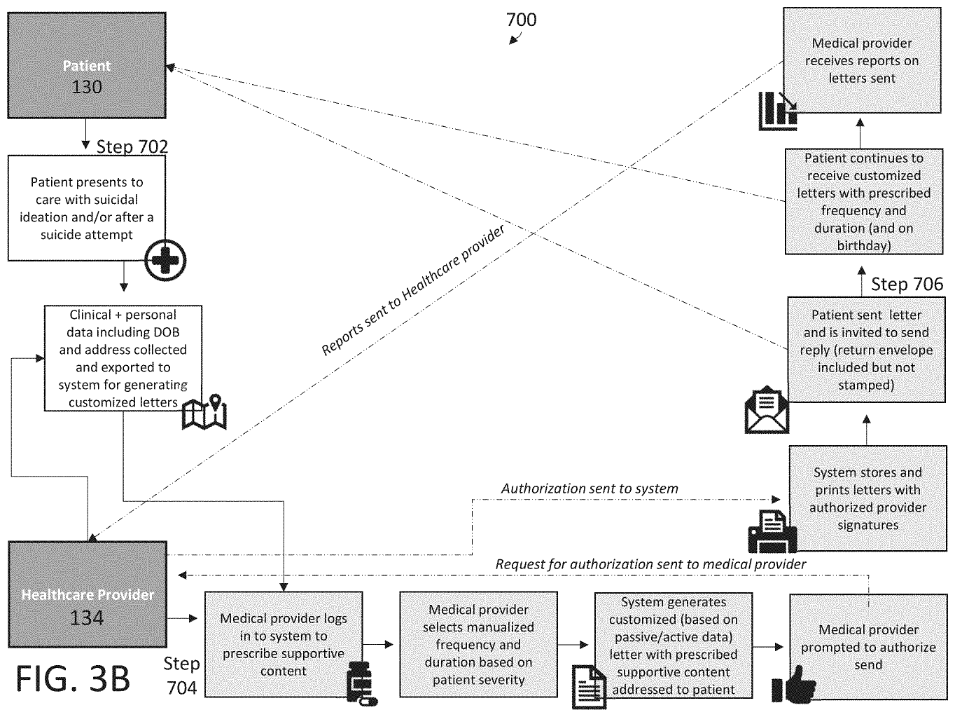

FIG. 3A is a flow chart illustrating exemplary computer-implemented method 300 for adapting and providing treatment to a patient suffering from a mental disorder or mental illness, according to some embodiments described herein. Method 300 includes obtaining patient data related to a mental condition of a patient at step 302, adapting treatment for the mental condition of the patient at step 304, and administering the treatment at step 306. The treatment may address the patient's mental condition, such as by addressing suicidal tendencies of a suicidal patient. In some embodiments, method 300 may be performed by one or more devices 120 illustrated in FIGS. 1A-2.

Obtaining patient data related to a mental condition of a patient at step 302 may include receiving the patient data over communication network 102 from computer 110 and/or other devices 120. In some embodiments, patient data may be obtained from the patient's electronic health records and/or via the patient's healthcare provider, such as at optionally included step 302a. For example, the patient data may include instructions for selecting a treatment activity and/or for generating a list of treatment activities. In another example, the patient data may further include personal data relating to the patient for use in generating personalized content (e.g., letters with supportive content, etc.), as described herein. In some embodiments, patient data may be obtained in the form of diagnosis data from the patient's healthcare provider, such as at optionally included step 302b. In some embodiments, patient data may indicate that the patient received ECT, intravenous ketamine, esketamine, any ketamine derivative or NDMA, glutamate modulating entity, psychedelic drugs, and/or rTMS treatment recently, such as within the last 24, 36, or 48 hours, or is currently doing so. In some embodiments the patient data may include an indication of treatment activities for selecting to administer. In some embodiments, obtaining patient data may include obtaining patient data from device 120, such as including sensory and/or application data from device 120. In one example, the patient data may be obtained by prompting the patient for manual input. In some embodiments, obtaining the patient data may include displaying a notification on a display of device 120 asking the patient whether the patient is ready for a treatment activity. In some embodiments, obtaining the patient data may include obtaining sensory data from one or more sensors of device 120. For example, the sensory data may indicate the patient's response to past treatment activity, and/or a current mental status of the patient. In some embodiments, obtaining the patient data may include accessing an application on device 120. For example, device 120 may determine a risk level of the patient based on words spoken by the patient (e.g., during a phone call), a message sent by the patient, a social media post, or other such activity.

Adapting treatment for the mental condition of the patient at step 304 may include selecting the treatment from a list of treatment activities, such as at optionally included step 304a. In some embodiments, adapting the treatment may include selecting treatment activities out of order from an ordered list. For example, a first treatment activity may be selected rather than a second treatment activity even though the second activity may be listed before the first treatment activity in the ordered list. In this example, the patient data obtained at step 302 may indicate the patient's readiness for the first treatment activity and/or indicate that the patient is not ready for the second treatment activity. The second treatment activity may be omitted from the list, or may be selected at a later time. In some embodiments, adapting treatment may include inputting patient data to a trained model and receiving an indication of one or more treatment activities as an output from the trained model such as at optionally included step 304b, such as described herein including in connection with system 100. In some embodiments, adapting treatment may include determining whether the patient received ECT, intravenous ketamine, esketamine, any ketamine derivative or NDMA, glutamate modulating entity, psychedelic drugs, and/or rTMS treatment and when, and scheduling or administering treatment immediately or within 24, 36, or 48 hours.

In some embodiments, method 300 may further include transmitting to the patient's healthcare provider an indication of the patient's response to the treatment activity, such as over communication network 102. In some embodiments, method 300 may further include accessing one or more applications on device 120 to determine a contact of the patient, and/or sending a message to the contact. For example, the message may include a status update of the patient. In some embodiments, the message may include a request that the contact check in with the patient.

In some embodiments, method 300 may further include generating a message template. The message template may be adapted to generate a message to send to the patient. For example, the message template may not initially include the patient's name or any information about the patient's condition until adapted for the patient. Rather, the message template may be generated (e.g., by computer 110 and/or device 120) in response to a particular event, and/or after a particular amount of time since the patient first checked in to a clinic. In some embodiments, method 300 may include sending the message on behalf of a healthcare provider of the patient. For example, the message may be sent in the name of the healthcare provider (e.g., clinician or group of clinicians and/or clinicians' assistants). In some embodiments, the message includes a request for the patient to provide a status update. For example, the message may ask the patient how the patient is feeling. In some embodiments, the message is signed by the healthcare provider. For example, the message may include a printed, signed, and scanned version of a letter. Alternatively, the message may include an automatically generated image of the healthcare provider's signature. In some embodiments, the frequency and/or duration may be set by the patient's healthcare provider.

In some embodiments, generating and sending messages to a patient from a provider may include generating message content and sending a message. In some embodiments, obtaining patient data may include obtaining personal and/or health related information for the patient. For example, the patient may check in for first-time care and provide the patient data. The information may include the patient's date of birth, address, and/or the patient's condition (e.g., if already known).

In some embodiments, generating message content may include the provider selecting content for messages. For example, the provider may select the content based on the mental condition of the patient and/or based on patient data obtained previously. In some embodiments, the provider may select a duration over which messages are to be sent, and/or the frequency at which the messages are to be sent. In some embodiments, computer 110 may automatically generate the messages using content selected by the provider. For example, computer 110 may generate the messages at the frequency set by the provider over the duration set by the provider. In some embodiments, the messages may be electronically signed and/or signed by hand prior to being sent. In some embodiments, the signed messages may be stored on computer 110.

In some embodiments, sending a message may include emailing and/or mailing one or more messages to the patient. For example, the messages may be sent at a frequency and duration set by the provider. In some embodiments, paper letters including the messages may be mailed to the address of the patient obtained previously. In some embodiments, the paper letter may be enclosed in an envelope with a return envelope included. For example, the patient may respond to the paper letter using the return envelope. In some embodiments, computer 110 may generate reports based on sent paper letters (e.g., frequency, duration, content, etc.) for the provider to review.

FIG. 3B is a flow chart illustrating exemplary method 700 for generating and sending messages to patient 130 from provider 134, according to some embodiments. Method 700 includes obtaining patient data at step 702, generating message content at step 704, and sending a message at step 706.

Obtaining patient data at step 702 may include obtaining personal and/or health related information for patient 130. For example, patient 130 may check in for first-time care and provide the patient data. The information may include the patient's date of birth, address, and/or the patient's condition (e.g., if already known). In some embodiments, the patient data obtained at step 702 may indicate that the patient received ECT, intravenous ketamine, esketamine, any ketamine derivative or NDMA, glutamate modulating entity, psychedelic drugs, and/or rTMS treatment recently, such as within the last 24, 36, or 48 hours, or is currently doing so.

Generating message content at step 704 may include provider 132 selecting content for messages. For example, provider 132 may select the content based on the mental condition of patient 130, and/or based on patient data obtained at step 702. In some embodiments, provider 132 may select a duration over which messages are to be sent, and/or the frequency at which the messages are to be sent. In some embodiments, computer 110 may automatically generate the messages using content selected by provider 134. For example, computer 110 may generate the messages at the frequency set by provider 134 over the duration set by provider 134. In some embodiments, the messages may be electronically signed and/or signed by hand prior to being sent. In some embodiments, the signed messages may be stored on computer 110.

Sending a message at step 706 may include emailing and/or mailing one or more messages to patient 130. For example, the messages may be sent at a frequency and duration set by provider 134. In some embodiments, paper letters including the messages may be mailed to the address of patient 130 obtained at step 702. In some embodiments, the paper letter may be enclosed in an envelope with a return envelope included. For example, patient 130 may respond to the paper letter using the return envelope. In some embodiments, computer 110 may generate reports based on sent paper letters (e.g., frequency, duration, content, etc.) for provider 134 to review.

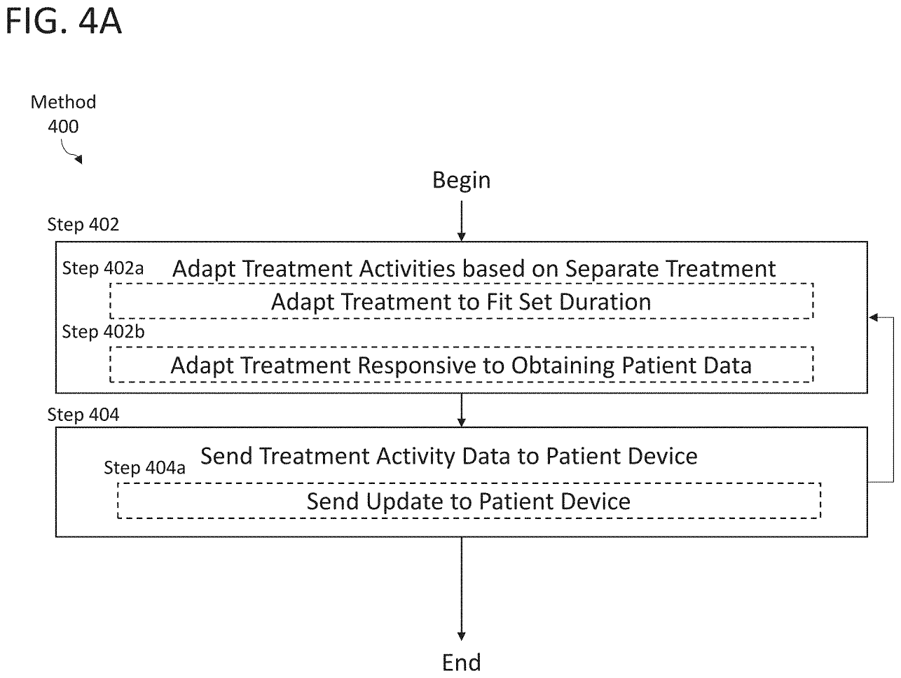

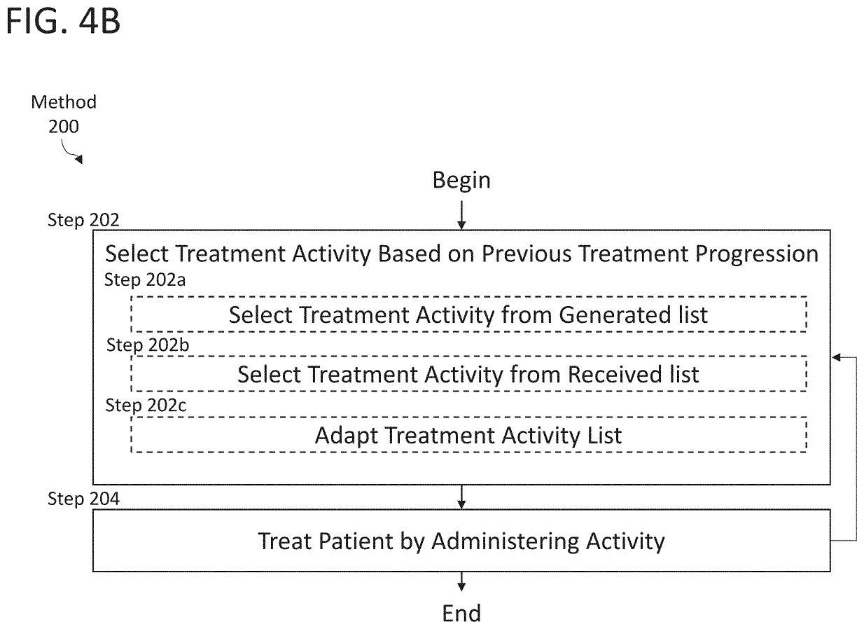

FIG. 4A is a flow chart illustrating exemplary method 400 for adapting and delivering personalized care to a patient's device, according to some embodiments described herein. Method 400 includes adapting a list of treatment activities to be administered to a patient at step 402, and sending the treatment activity data indicative of the list of treatment activities over a communication network to the patient's device at step 404. In some embodiments, method 400 may be performed by computer 110 and/or device 120 illustrated in FIGS. 1A-2. According to various embodiments, method 400 may be implemented in an inpatient or outpatient setting, as described herein

Adapting the list of treatment activities at step 402 may include adapting the list of treatment activities to fit a duration of treatment, such as at optionally included step 402a. For example, the patient may be treated inpatient for a week, and the list of treatment activities may be adapted to fit the week. For inpatient implementation, patient data from the patient's provider and/or clinician may be incorporated in adapting the list of treatment activities, as may be sensory and/or application data from the patient's device. In some embodiments, treatment steps may be added, omitted, and/or swapped in order of administration to fit the duration of treatment. For outpatient implementation, patient data may further include contact information for a contact of the patient to notify of the patient's status and/or coordinate interaction. In some embodiments, adapting the list of treatment activities may be responsive to obtaining patient data, such as at optionally included step 402b. For example, in some embodiments, the patient data may be received over communication network 102, from device 120. The patient data may be indicative of the patient's response to one or more previous treatment activities. In some embodiments, the list of treatment activities may be adapted based on the patient data. In some embodiments, the list of treatment activities may be adapted based on the patient's electronic health records.

Sending the treatment activity data at step 404 may include sending an update to the list of treatment activities on device 120, such as at optionally included step 404a. For example, instructions may be sent detailing steps to add, remove, and/or reorder from an existing list. Alternatively, in some embodiments, sending the list may include sending a new list of treatment activities, such as to replace the existing list.

In some embodiments, method 400 may further include sending a message to a healthcare provider of the patient. For example, the message may relate to the patient, such as including a status update of the patient's mental condition, the patient's response to previous treatment, and/or a notification that the patient is at risk of dying by suicide.

FIG. 4B is a flow chart illustrating exemplary computer-implemented method 200 for treating a patient who is at risk of dying by suicide, according to some embodiments described herein. Method 200 includes selecting at least one treatment activity from a list at step 202 and treating a patient to prevent suicide by administering, to the patient, the treatment activity at step 204. In some embodiments, the list may include at least one of: an interactive experience tracking module (such as a diary) tracking at least one metric related to behavior of the patient; instructions on modifying behavior of the patient; information regarding stimulus control; relaxation training; interactive multimedia content for paced breathing, progressive muscle relaxation, imagery-induced relaxation, and/or self-hypnosis; instructions on use of medication; and/or instructions on user monitoring of and adjustment of thoughts of the patient. In some embodiments, method 200 may be performed by one or more devices 120 illustrated in FIGS. 1A-2.

Selecting at least one treatment activity from the list at step 202 may include generating a list of treatment activities at step 202a and/or receiving a list of treatment activities over communication network 102 at step 202b. For example, in some embodiments, computer 110 may generate and send the list over communication network 102 to device(s) 120. In some embodiments, the list of treatment activities may be generated and/or adapted at step 202c based on patient data obtained from the patient's electronic health records, sensory data collected by device(s) 120, manual input from the patient, and/or instructions from the patient's healthcare provider. In some embodiments, selecting the treatment activity from the list does not include generating or adapting the list. For example, in some embodiments, device(s) 120 may have an up-to-date list upon performing step 202. In some embodiments, selecting the treatment activity may include selecting the next treatment activity from the list based on an order of the list. In some embodiments, the list may include CBT steps.

In some embodiments, method 200 may further include sending, to a healthcare provider of the patient, a message. For example, in some embodiments, the message may indicate a status of the patient. In some embodiments, the message may notify the healthcare provider that the patient is at risk of suicide. In some embodiments, the message may provide the healthcare provider with suggested discussion items for upcoming meetings with the patient.

In some embodiments, method 200 may further include generating a message template. The message template may be adapted to generate a message to send to the patient. For example, the message template may not initially include the patient's name or any information about the patient's condition until adapted for the patient. Rather, the message template may be generated (e.g., by computer 110 and/or device 120) in response to a particular event, and/or after a particular amount of time since the patient first checked in to a clinic. In some embodiments, method 200 may include sending the message on behalf of a healthcare provider of the patient. For example, the message may be sent in the name of the healthcare provider (e.g., clinician or group of clinicians and/or clinicians' assistants). In some embodiments, the message may include a request for the patient to provide a status update. For example, the message may ask the patient how the patient is feeling. In some embodiments, the message may be signed by the healthcare provider. For example, the message may include a printed, signed, and scanned version of a letter. Alternatively, the message may include an automatically generated image of the healthcare provider's signature.

In some embodiments, method 200 may further include recording a suicidal episode of the patient. In some embodiments, recording the suicidal episode may include capturing audio and/or video of the suicidal episode. For example, the recording may be performed by device 120 (e.g., a mobile phone and/or personal computing device of the patient). In some embodiments, recording the suicidal episode may include a written narrative of the suicidal episode. In some embodiments, the narrative may be provided manually (e.g., in spoken, written, and/or typed form) by the patient. In some embodiments, suicidal thoughts and/or behaviors may be captured in the recording of the suicidal episode.

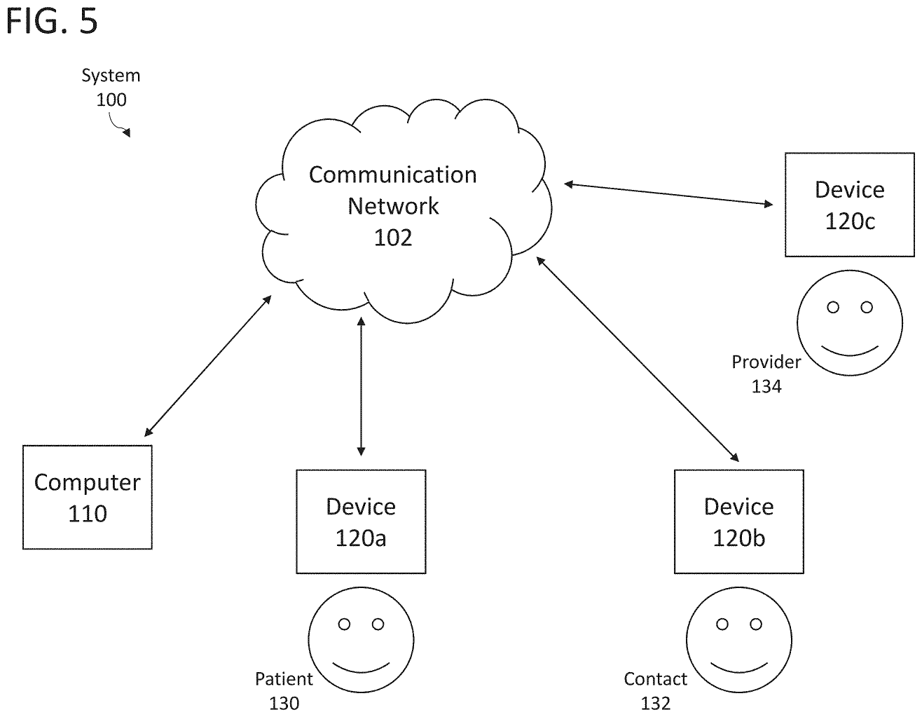

FIG. 5 is a block diagram of system 100 further illustrating interactivity between patient 130, contact 132 of the patient, and the patient's healthcare provider 134 via the system. In FIG. 5, device 120a is a device of patient 130, device 120b is a device of contact 132, and device 120c is a device of provider 132. For example, devices 120a-120c may include mobile phones, tablet computers, desktop and/or laptop computers, and/or other such devices. Computer 110 may include a server and/or any other suitable device or system.

In some embodiments, device 120a may obtain contact information for contact 132 and provide the contact information to computer 110. For example, in some embodiments, computer 110 may send a message to contact 132 requesting contact 132 check in with patient 130. In some embodiments, computer 110 may be configured to coordinate sending notifications to specified contact devices based on application data received from the patient's device 120. In one example, application data received from the patient's device 120 may indicate it has been at least a threshold amount of time since the patient's device received a call or message from contact device 120b. In this example, computer 110 may send a notification to contact device 120 to be displayed for contact 132 asking whether contact 132 would like to reach out to the patient. In some embodiments, notifications to contacts from computer 110 may include educational messages explaining the benefits of receiving messages from a contact.

In some embodiments, device 120a may indicate a status of patient 130 to computer 110 over communication network 102. Computer 110 may communicate the status to device 120c of provider 134. Alternatively or additionally, computer 110 may communicate the status to device 120b of contact 132. In some embodiments devices 120a-120c may communicate directly to one another, such as within a decentralized system which does not include computer 110. In some embodiments, if patient data (e.g., application data, sensory data, etc.) received from device 120a indicates the patient is at risk, computer 110 may be configured to send a notification to device 120b or device 120c such that contact 132 and/or provider 134 can contact the patient.

Exemplary Results: The following results indicate the effectiveness of techniques described herein and include exemplary embodiments.

The inventor used some embodiments herein (such as selecting treatment activity from a list including CBT; administering personalized, adaptive treatment; and/or coordinating treatments for patients, connecting patients to providers, connecting patients to contacts, and adapting treatment based on information from healthcare providers and the patients' electronic health records, etc.) on several patients who had recently received intensive acute treatment, such as the acute treatments described herein. After the intensive acute treatment, more than half of these patients still had specific thoughts related to acting on suicide ideation. However, after using some embodiments herein, one third of these patients went from having such specific thoughts to having none over the subsequent 4-8 weeks.

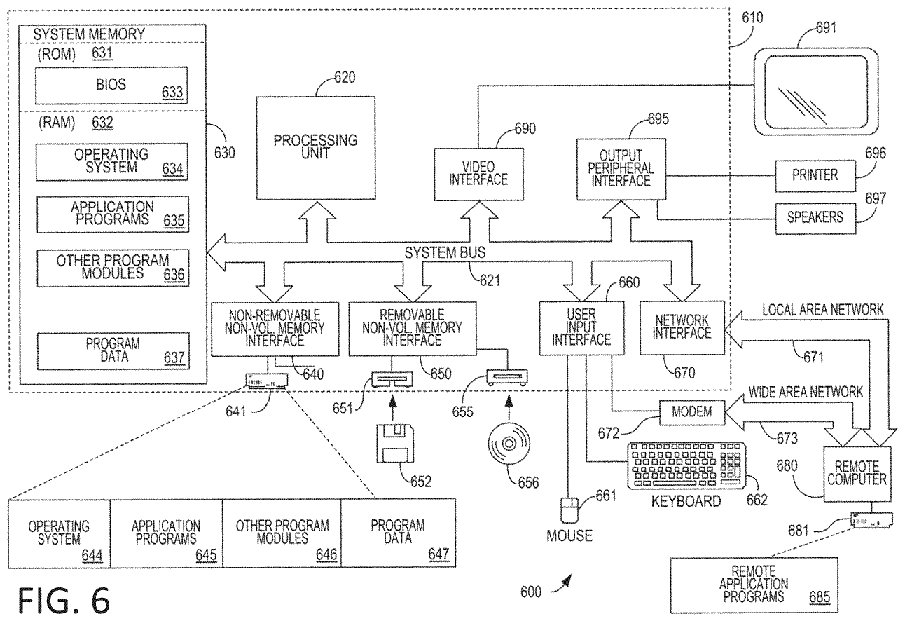

FIG. 6 illustrates an example of a suitable computing system environment 600 on which some embodiments may operate. The computing system environment 600 is only one example of a suitable computing environment and is not intended to suggest any limitation as to the scope of use or functionality of the application. Neither should the computing environment 600 be interpreted as having any dependency or requirement relating to any one or combination of components illustrated in the exemplary operating environment 600.

Some embodiments are operational with numerous other general purpose or special purpose computing system environments or configurations. Examples of well-known computing systems, environments, and/or configurations that may be suitable include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The application may also be practiced in distributed computing environments where tasks are performed by remote processing devices that are linked through a communications network. In a distributed computing environment, program modules may be located in both local and remote computer storage media including memory storage devices.

With reference to FIG. 6, an exemplary system for implementing embodiments includes a general purpose computing device in the form of a computer 610. In some embodiments, computer 610 may be dedicated to a particular task, although it may be a computer that would, in normal operation, store or retrieve information from a storage device.

Components of computer 610 may include, but are not limited to, a processing unit 620, a system memory 630, and a system bus 621 that couples various system components including the system memory to the processing unit 620. The system bus 621 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 610 typically includes a variety of computer readable media. Computer readable media can be any available media that can be accessed by computer 610 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 610. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer readable media.

The system memory 630 includes computer storage media in the form of volatile and/or nonvolatile memory such as read only memory (ROM) 631 and random access memory (RAM) 632. A basic input/output system 633 (BIOS), containing the basic routines that help to transfer information between elements within computer 610, such as during start-up, is typically stored in ROM 631. RAM 632 typically contains data and/or program modules that are immediately accessible to and/or presently being operated on by processing unit 620. By way of example, and not limitation, FIG. 6 illustrates operating system 634, application programs 635, other program modules 636, and program data 637.

The computer 610 may also include other removable/non-removable, volatile/nonvolatile computer storage media. By way of example only, FIG. 6 illustrates a hard disk drive 641 that reads from or writes to non-removable, nonvolatile magnetic media, a magnetic disk drive 651 that reads from or writes to a removable, nonvolatile magnetic disk 652, and an optical disk drive 655 that reads from or writes to a removable, nonvolatile optical disk 656 such as a CD-ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 641 is typically connected to the system bus 621 through an non-removable memory interface such as interface 640, and magnetic disk drive 651 and optical disk drive 655 are typically connected to the system bus 621 by a removable memory interface, such as interface 650.

The drives and their associated computer storage media discussed above and illustrated in FIG. 6, provide storage of computer readable instructions, data structures, program modules and other data for the computer 610. In FIG. 6, for example, hard disk drive 641 is illustrated as storing operating system 644, application programs 645, other program modules 646, and program data 647. Note that these components can either be the same as or different from operating system 634, application programs 635, other program modules 636, and program data 637. Operating system 644, application programs 645, other program modules 646, and program data 647 are given different numbers here to illustrate that, at a minimum, they are different copies. A patient or other user may enter commands and information into the computer 610 through input devices such as a keyboard 662 and pointing device 661, commonly referred to as a mouse, trackball, or touch pad. Other input devices (not shown) may include a microphone, joystick, game pad, satellite dish, scanner, or the like. These and other input devices are often connected to the processing unit 620 through a user input interface 660 that is coupled to the system bus, but may be connected by other interface and bus structures, such as a parallel port, game port or a universal serial bus (USB). A monitor 691 or other type of display device is also connected to the system bus 621 via an interface, such as a video interface 690. In addition to the monitor, computers may also include other peripheral output devices such as speakers 697 and printer 696, which may be connected through an output peripheral interface 695.

The computer 610 may operate in a networked environment using logical connections to one or more remote computers, such as a remote computer 680. The remote computer 680 may be a personal computer, a server, a router, a network PC, a peer device or other common network node, and typically includes many or all of the elements described above relative to the computer 610, although only a memory storage device 681 has been illustrated in FIG. 6. The logical connections depicted in FIG. 6 include a local area network (LAN) 671 and a wide area network (WAN) 673, but may also include other networks. Such networking environments are commonplace in offices, enterprise-wide computer networks, intranets, and the Internet.

When used in a LAN networking environment, the computer 610 is connected to the LAN 671 through a network interface or adapter 670. When used in a WAN networking environment, the computer 610 typically includes a modem 672 or other means for establishing communications over the WAN 673, such as the Internet. The modem 672, which may be internal or external, may be connected to the system bus 621 via the user input interface 660, or other appropriate mechanism. In a networked environment, program modules depicted relative to the computer 610, or portions thereof, may be stored in the remote memory storage device. By way of example, and not limitation, FIG. 6 illustrates remote application programs 685 as residing on memory device 681. It will be appreciated that the network connections shown are exemplary and other means of establishing a communications link between the computers may be used.

Having thus described several aspects of at least one embodiment of this application, it is to be appreciated that various alterations, modifications, and improvements will readily occur to those skilled in the art.

Such alterations, modifications, and improvements are intended to be part of this disclosure, and are intended to be within the spirit and scope of the application. Further, though advantages of the present application are indicated, it should be appreciated that not every embodiment will include every described advantage. Some embodiments may not implement any features described as advantageous herein and in some instances. Accordingly, the foregoing description and drawings are by way of example only.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semicustom or custom. As a specific example, some commercially available microprocessors have multiple cores such that one or a subset of those cores may constitute a processor. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the application may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the application discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present application as discussed above. As used herein, the term “computer-readable storage medium” encompasses only a computer-readable medium that can be considered to be a manufacture (i.e., article of manufacture) or a machine. Alternatively or additionally, the application may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present application as discussed above. Additionally, it should be appreciated that according to one aspect of this embodiment, one or more computer programs that when executed perform methods of the present application need not reside on a single computer or processor, but may be distributed in a modular fashion amongst a number of different computers or processors to implement various aspects of the present application.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

Various aspects of the present application may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the application may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. A non-transitory computer-readable storage medium having encoded thereon instructions that, when executed by at least one processor, cause the at least one processor to carry out a method, the method comprising:
obtaining patient data related to a mental condition of a patient, wherein the patient data indicates timing information related to previously administered treatment for the mental condition of the patient;
adapting, based on the patient data, a treatment for the mental condition of the patient, including adapting a rate or order of the treatment; and
administering, to the patient, the treatment.

2. The non-transitory computer-readable storage medium of claim 1, wherein the timing information related to the previously administered treatment indicates that, within 48 hours before administering the treatment to the patient, the patient received a previous treatment from a previous treatment list, the previous treatment list including:
electroconvulsive therapy;
intravenous ketamine;
esketamine;
a ketamine derivative;
N-Methyl-D-aspartate;
glutamate modulating entity;
psychedelic drugs; and
repetitive transcranial magnetic stimulation.

3. The non-transitory computer-readable storage medium of claim 1, wherein:
the timing information related to the previously administered treatment indicates the previous progression of the patient through the previously administered treatment; and
adapting the rate or order of the treatment comprises adapting the rate or order of the treatment based on the previous progression of the patient through the previously administered treatment.

4. The non-transitory computer-readable storage medium of claim 1, wherein adapting the rate or order of the treatment comprises:
selecting, from an ordered list of treatment activities, at least one first treatment activity, rather than selecting at least one second treatment activity listed before the at least one first treatment activity in the ordered list; and
selecting, at a later time, the at least one second treatment activity.

5. The non-transitory computer-readable storage medium of claim 1, wherein:
obtaining the patient data comprises obtaining sensory data from one or more sensors of a device of the patient; and
the patient data indicates a response of the patient to the previously administered treatment.

6. The non-transitory computer-readable storage medium of claim 1, wherein:
obtaining the patient data comprises asking the patient whether the patient is ready for a treatment activity; and
adapting the treatment comprises selecting the treatment activity from a list of treatment activities.

7. The non-transitory computer-readable storage medium of claim 1, wherein:
obtaining the patient data comprises obtaining, over a communication network, instructions for selecting a treatment activity from a list of treatment activities;
adapting the treatment comprises selecting the treatment activity; and
transmitting, over the communication network to the healthcare provider, an indication of the patient's response to the treatment activity.

8. The non-transitory computer-readable storage medium of claim 1, wherein obtaining the patient data comprises:
accessing an application on a device of the patient; and
determining a risk of suicide of the patient based on one or more of:
words spoken by the patient; or
a message sent by the patient.

9. The non-transitory computer-readable storage medium of claim 8, wherein:
accessing the application comprises determining a contact of the patient; and
the method further comprises sending, to the contact, a message.

10. A system comprising at least one processor configured to:
based on information indicating the patient received, within 48 hours before a treatment activity of a list of treatment activities to be administered to a patient is administered to the patient, a separate treatment from an additional list:
adapt, for a mental condition of the patient, the list of treatment activities to be administered to the patient; and
send, over a communication network, to a device of the patient, treatment activity data indicating the list of treatment activities.

11. The system of claim 10, wherein the additional list includes:
electroconvulsive therapy;
intravenous ketamine;
esketamine;
a ketamine derivative;
N-Methyl-D-aspartate;
glutamate modulating entity;
psychedelic drugs; and
repetitive transcranial magnetic stimulation.

12. The system of claim 10, wherein the at least one processor is configured to adapt the list of treatment activities to fit a duration of inpatient treatment of the patient.

13. The system of claim 10, wherein the at least one processor is further configured to:
obtain, over the communication network, from the device, patient data indicative of the patient's response to at least one treatment activity of the list of treatment activities, including previous progression of the patient through the at least one treatment activity;
adapt, based on the patient data, the list of treatment activities, including adapting a rate or order of the list of treatment activities based on the previous progression of the patient; and
send, over the communication network, to the device, an update to the treatment activity data.

14. The system of claim 10, wherein the at least one processor is further configured to:
access electronic health records of the patient; and
adapt the list of treatment activities based on the electronic health records.

15. The system of claim 10, wherein the at least one processor is further configured to:
send, to a healthcare provider of the patient, a first message relating to the patient; and
send, to a contact of the patient, a second message relating to the patient.

16. A non-transitory computer-readable storage medium having encoded thereon instructions that, when executed by at least one processor, cause the at least one processor to carry out a method, the method comprising:
based on previous progression of a patient through a previous treatment, selecting at least one treatment activity from a first list, the first list including:
an interactive experience tracking module configured to track at least one metric related to behavior of the patient;
instructions on modifying behavior of the patient;
information regarding stimulus control;
relaxation training;
interactive multimedia content for paced breathing, progressive muscle relaxation, imagery-induced relaxation, and/or self-hypnosis;
instructions on use of medication; and
instructions on user monitoring of and adjustment of thoughts of the patient; and
treating the patient by administering, to the patient, the at least one treatment activity within 48 hours of administering at least one other treatment from a second list.

17. The non-transitory computer-readable storage medium of claim 16, wherein the second list includes:
electroconvulsive therapy;
intravenous ketamine;
esketamine;
a ketamine derivative;
N-Methyl-D-aspartate;
glutamate modulating entity;
psychedelic drugs; and
repetitive transcranial magnetic stimulation.

18. The non-transitory computer-readable storage medium of claim 16, wherein:
the at least one treatment activity is occurring within 36 hours of administering the at least one separate treatment.

19. The non-transitory computer-readable storage medium of claim 16, wherein:
selecting the at least one treatment activity comprises selecting a cognitive behavioral therapy (CBT) step from the first list; and
treating the patient comprises administering the CBT step to the patient.

20. The non-transitory computer-readable storage medium of claim 16, wherein the method further comprises sending, to a healthcare provider of the patient, a message notifying the healthcare provider that the patient is at risk of suicide.
